(19) United States
(12) Patent Application Publication
(10) Pub. No.: US 2025/0319654 A1
(43) Pub. Date: Oct. 16, 2025

(54) PROCESS FOR PRODUCING A THREE-DIMENSIONAL STRUCTURE

(71) Applicant: MATERIAS S.R.L., Napoli (IT)

(72) Inventors: Selene DE MARTINO, Piano di Sorrento (Napoli) (IT); Mario BATTISTI, Ercolano (Napoli) (IT); Aniello CAMMARANO, Ascea (Salerno) (IT); Luigi NICOLAIS, Ercolano (Napoli) (IT)

(21) Appl. No.: 18/863,187

(22) PCT Filed: May 5, 2023
(86) PCT No.: PCT/IB2023/054678

§ 371 Date: Nov. 5, 2024

(30) Foreign Application Priority Data
May 6, 2022 (IT) 102022000009341

(51) Int. Cl. B29C 64/135; B29C 64/277; B33Y 10/00

(52) U.S. Cl. CPC B29C 64/135; B29C 64/277; B33Y 10/00

(57) ABSTRACT

A process for producing a three-dimensional structure involves providing a light source, coupling the light source to a proximal end of an optical fibre to propagate a light generated by the light source through the optical fibre and produce, at a distal end of the optical fibre, a predetermined incident optical field, providing a photo-crosslinkable polymeric material coated with a transparent material having a first surface in contact with the photo-crosslinkable polymeric material and a second surface not in contact with the photo-crosslinkable polymeric material and opposite to the first surface, placing the distal end of the optical fibre at a distance D from the second surface of the transparent material, and irradiating the second surface of the transparent material with the light propagated and exiting the distal end of the optical fibre, obtaining propagation of the light through the transparent material and photo-crosslinking, by irradiation, of the polymeric material.

### FIELD OF THE INVENTION

The present invention relates to a process for producing a three-dimensional structure, the three-dimensional structure thus obtained, and uses thereof.

### STATE OF THE ART

The phenomenon of the interaction between light (understood as a portion of the electromagnetic spectrum visible to the human eye) and matter (understood as any object that has mass and occupies space) is usually used, in different ways, for the manufacture of three-dimensional structures having different dimensional scales.

For example, two-photon confocal microscopy is used for the manufacture of nanometer-scale structures by cross-linking photosensitive materials irradiated with coherent light (as well-known to the person skilled in the art, in optics, coherence—or phase coherence—designates the property of an electromagnetic wave to maintain a certain phase relationship with itself during its propagation).

Photolithography, on the other hand, uses ultraviolet light and chrome-on-glass masks to create two-dimensional circuits on photoresists ranging in size from nanometers to millimeters.

Recently, a process for the manufacture of microneedles made of polymeric material using standard photolithography and acrylate polymers mixed with a photo-crosslinking material has been patented (CA3061448A1). This method, while having advantages over traditional photolithographic techniques, is severely limited both by the need to change the mask whenever the geometric configuration and shape of the microneedles are to be changed, and by the type of material that can be used since it must guarantee that the obtained microneedles have excellent mechanical properties; furthermore, this method does not allow the manufacture of three-dimensional structures (such as for example microneedles) on curved surfaces—the flatness of the surface on which the three-dimensional structures are manufactured, in fact, is a prerequisite for the photolithographic method of the prior art.

Therefore, there is a need in the market for a process for the manufacture of microneedles, and generally of three-dimensional structures, which does not require the use of a photolithographic mask or other optical component (such as for example lenses, diaphragms, pinhole lenses), in general, or whenever the geometric configuration and shape of the desired microneedles (or in general the desired three-dimensional structures) are to be changed, and which uses a type of material capable of guaranteeing that the obtained microneedles/three-dimensional structures have excellent mechanical properties and allows the manufacture of three-dimensional structures (such as for example microneedles) on curved surfaces.

### SUMMARY OF THE INVENTION

The object of the present invention is therefore to provide a process for the manufacture of microneedles, and generally of three-dimensional structures, which does not require the use of a photolithographic mask or other optical component (such as for example lenses, diaphragms, pinhole lenses), in general, or whenever the geometric configuration, geometric arrangement, and shape of the desired microneedles (or in general the desired three-dimensional structures) are to be changed, which uses a type of material capable of guaranteeing that the obtained microneedles/three-dimensional structures have excellent mechanical properties and allows the manufacture of three-dimensional structures (such as for example microneedles) on curved surfaces.

This object is achieved by means of a process for producing a three-dimensional structure as outlined in the appended claims, the definitions of which form an integral part of the present specification.

### BRIEF DESCRIPTION OF THE FIGURES

The invention will be better understood by referring to the following detailed description of preferred embodiments thereof, which is provided by way of non-limiting example with reference to the attached figures, wherein:

FIG. 1 shows the trend of the fundamental mode of light leaving an optical fibre.

FIG. 2 shows a summary diagram of the elements of the production process according to the present invention.

FIG. 3 shows three-dimensional structures, in particular needles, obtained with the process according to the present invention.

FIG. 4 shows three-dimensional structures obtained with the process according to the present invention.

FIG. 5 shows possible geometries achievable by changing the mode field shape of the light propagated in the optical fibre according to the process of the present invention.

FIG. 6 shows a schematic representation of the movement of the optical fibre along a curved surface in the process according to the present invention.

FIG. 7 shows three-dimensional conical structures obtained on curved surfaces by the process according to the present invention.

In the attached figures, the same or similar elements will be indicated by the same numerical references.

### DETAILED DESCRIPTION OF THE INVENTION

A first object of the present invention relates to a process for producing a three-dimensional structure comprising the steps of:
a) providing a light source;
b) coupling said light source to a proximal end of at least one optical fibre, in such a way as to propagate the light generated by said light source through the optical fibre and produce, at a distal end of the optical fibre, thus at the outlet of the optical fibre, a predetermined incident optical field, wherein said optical field maintains the same phase profile, the same spatio-temporal intensity, and the same frequency during propagation of the light through the optical fibre and its exit from the optical fibre;
c) providing at least one photo-crosslinkable polymeric material coated with at least one transparent material, wherein the transparent material comprises a first surface placed in contact with the photo-crosslinkable polymeric material, and a second surface placed not in contact with the photo-crosslinkable polymeric material and opposite to the first surface;
d) placing the distal end of the optical fibre at a distance D from the second surface of the transparent material, where D ranges from 0 mm to 5 mm;
e) irradiating the second surface of the transparent material with the light propagated and exiting the distal end of the optical fibre for a period of time between 1 second and 5 minutes, so as to obtain propagation of the light through the transparent material towards the photo-crosslinkable polymeric material and to obtain photo-crosslinking, by irradiation, of the polymeric material thus irradiated, with consequent formation of the three-dimensional structure.

Advantageously, in order to overcome the shortcomings described above, the present invention thus developed a process capable of producing three-dimensional structures made of photo-crosslinkable polymeric material by using the properties of light propagated in an optical fibre.

As well-known to the person skilled in the art, optical fibres are devices commonly used in the telecommunications industry. They are commonly made up of two materials, which have different refractive indexes and can enable the propagation of luminous radiation (light) even at a distance of several kilometres, while maintaining the characteristics of the light beam, in particular the field profile and polarization, due to the principle of total internal reflection.

As well-known to the person skilled in the art, in summary, an optical fibre can preferably be pictured as a cable made up of very fine transparent filaments made of glass fibre or other plastic materials, held together in a small sheath of insulating material. Each individual filament is preferably composed of two concentric layers of transparent and extremely pure material: a central cylinder—the core—and a mantle—the so-called cladding —which covers it. The core has a diameter that can range from a few μm to several hundreds of μm, while the cladding preferably has a diameter of approximately 125 μm. The optical fibre is preferably sheathed in a protective sock called jacket, which protects the fibre from physical stress, on the one hand, and from corrosion, on the other hand.

As is well known, inside an optical fibre the so-called guided modes propagate, the latter being stationary solutions obtained by solving Maxwell's equations for the electromagnetic field. Each guided mode has its own unique shape which can be complicated as desired, also considering the polarization of the coupled light entering the optical fibre. Therefore, the beneficial use of these peculiar properties of the guided modes and of the process steps according to the present invention allows the production of three-dimensional structures on photo-crosslinkable polymeric materials.

According to a preferred embodiment of the process according to the present invention, the at least one transparent material is preferably selected from: transparent natural polymer, transparent synthetic polymer, transparent pre-polymerized photo-crosslinked polymer, transparent polyethylene terephthalate, transparent polypropylene, transparent glass, transparent hydrogel, transparent silicone hydrogel. Furthermore, the first surface of the transparent material is preferably a curved surface. Preferably, therefore, the first surface of the transparent material has a radius of curvature between 0 (point surface) and infinite (flat surface). Even more preferably, it is a curved surface with a radius of curvature between 8.3 mm and 9 mm.

Still according to a preferred embodiment of the process according to the present invention, the photo-crosslinkable polymeric material preferably comprises: at least one photo-crosslinkable biocompatible hydrogel or mixtures thereof, at least one photoinitiator and optionally at least one non-photo-crosslinkable element.

Still according to a preferred embodiment of the process according to the present invention, the at least one photo-crosslinkable biocompatible hydrogel preferably comprises at least one of: hyaluronic acid acrylate derivatives, hyaluronic acid acrylate, hyaluronic acid methacrylate, acrylate or methacrylate gelatin derivatives, gelatin-methacryloyl (GelMA), or mixtures thereof.

Still according to a preferred embodiment of the process according to the present invention, the at least one photo-crosslinkable biocompatible hydrogel preferably further comprises at least one of: di- or tetra-acrylate cross-linker, 2- or 4-arm acrylate (polyethylene glycol diacrylate (PEGDA), 4-arm PEG-Acrylate, glycerol 1,3-diglycerolate diacrylate, tetra(ethylene glycol) diacrylate (TTEGDA), di(ethylene glycol) diacrylate, bisphenol A glycerolate (1-glycerol/phenol) diacrylate, 1,6-hexanediol ethoxylate diacrylate, tricyclo[5. 2.1.02,6]decanedimethanol diacrylate).

Still according to a preferred embodiment of the process according to the present invention, the at least one photoinitiator is preferably selected from: 2-hydroxy-2-methylpropiophenone, 2-hydroxy-4′-(2-hydroxyethoxy)-2-methylpropiophenone, lithium phenyl-2,4,6-trimethylbenzoylphosphinate (LAP).

Still according to a preferred embodiment of the process according to the present invention, the at least one non-photo-crosslinkable element is preferably selected from: silk fibroin, silica nanoparticle, titania nanoparticle, zirconia nanoparticle, gold nanoparticle, silver nanoparticle, zinc nanoparticle, substance having the biological activity to act as a pharmaceutical active ingredient, functionalized nanoparticle, free protein, protein covalently linked to hyaluronic acid methacrylate, substance having the biological activity to act as a pharmaceutical active ingredient included in a poly(lactic-co-glycolic acid) microsphere.

Preferably, in step e) of the process according to the present invention, the irradiation time is between 10 seconds and 60 seconds. Even more preferably, it is between 15 seconds and 45 seconds. Still more preferably, the irradiation time is 30 seconds.

Preferably, in step d) of the process according to the present invention, the distance D is between 0 mm and 1 mm.

Preferably, in step b) of the process according to the present invention, the light source is coupled to a proximal end of a bundle of optical fibres, wherein said bundle comprises a number of optical fibres comprised between 2 and 1000, wherein in said bundle the optical fibres are arranged at a mutual distance comprised between 0 mm and 10 mm, so as to control the interference of the relative optical fields of each optical fibre.

Within the present description and the present claims, the term “three-dimensional structure” refers to any structure having a three-dimensional shape; preferably having a shape selected from the following: cone, hollow cone, pyramid, hollow pyramid, needle, hollow needle, parallelepiped, hollow parallelepiped, cube, hollow cube, prism, hollow prism, polyhedron, hollow polyhedron.

Within the present description and the present claims, the term “light source” refers to any element capable of emitting the portion of the electromagnetic spectrum, visible to the human eye, between 400 nm and 700 nm wavelength, i.e., between 790 THz and 434 THz frequency, and/or the portion of the electromagnetic spectrum having a wavelength less than 400 nm.

As well-known to the person skilled in the art, the term “cross-linking” refers to a process by which polymer chains undergo a reaction that creates bonds (called cross-links) be tween different chains (or possibly between two different points in the same chain), at reactive functional groups. These bonds can be covalent or ionic, i.e., so-called strong bonds.

As well-known to the person skilled in the art, photo-crosslinking uses the presence of electromagnetic waves to trigger a cross-linking reaction.

The photo-crosslinkable polymeric material of the process according to the present invention is, advantageously, a polymeric material which, when irradiated by light, allows the triggering of a cross-linking reaction between the polymer chains of the polymer.

Within the present description and the present claims, the term “transparent material” refers to a material that can be crossed, throughout its thickness, by luminous electromagnetic radiation having a wavelength of interest (such as for example light), without causing substantial variation or deformation of the luminous electromagnetic radiation as it passes through the material; that is, a material which neither absorbs nor scatters the luminous electromagnetic radiation having a wavelength of interest which passes through the material. The luminous electromagnetic radiation will therefore be substantially the same and will substantially retain its physical and chemical properties both at the entrance and at the exit of the material it passes through.

Within the present description and the present claims, the term “hydrogel” refers to a colloid formed by polymeric chains of molecules dispersed in water, whose content of aqueous medium may exceed 99%.

Within the present description and the present claims, the term “photoinitiator” refers to a substance which, when exposed to UV light (e.g., light), generates a chemical species that triggers cross-linking reactions in the aforementioned photo-crosslinkable polymer.

According to a preferred embodiment of the process according to the present invention, the light generated by a light source (preferably selected from: halogen lamp, thermal lamp, LED, laser) is coupled to an optical fibre, thus allowing the propagation of light within the optical fibre, according to the well-known principles of geometric optics.

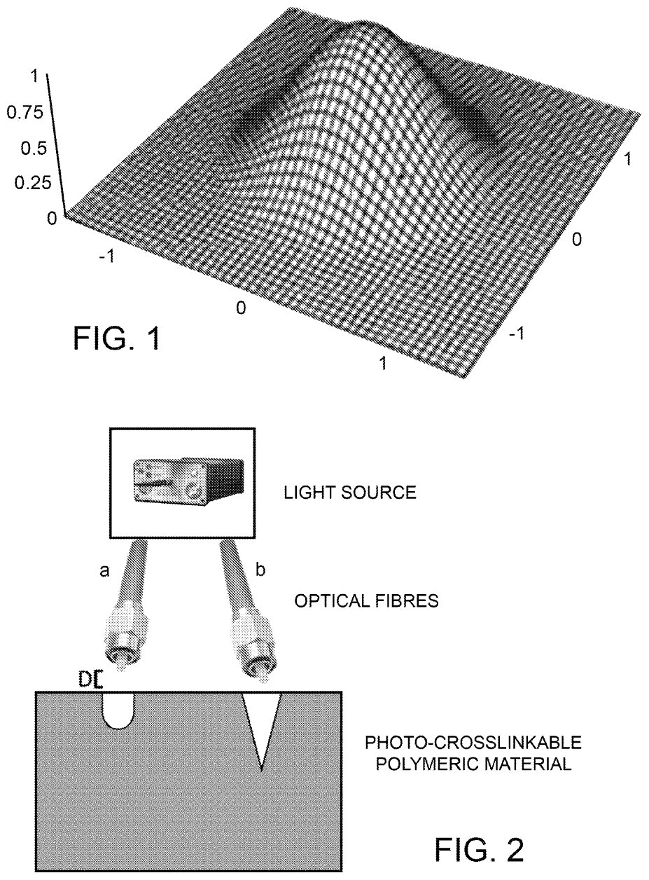

As well-known to the person skilled in the art, at the outlet of the optical fibre, the intensity profile of the electric field of the light (a quantity proportional to the power carried by the light) is three-dimensional and has an appearance like the one depicted in FIG. 1.

The field profile of the fundamental mode, called the Gaussian profile due to its bell shape, is advantageously sent onto a photo-crosslinkable polymeric material resulting in the formation of three-dimensional structures whose size and shape can be controlled by varying the irradiation time and the distance D between the surface of the material and the distal end of the optical fibre. The irradiation time is obviously proportional to the energy transferred to the material, whereas the variation of the distance D allows a variation in the width of the base of the three-dimensional structure.

FIG. 2 shows a summary diagram of the process for producing three-dimensional structures according to the present invention, as the D varies between the optical fibre and the surface: in FIG. 2b the distance D is 0 mm (a contact optical fibre), whereas in FIG. 2a the distance D is 5 mm (a non-contact optical fibre). FIG. 2b shows a diagram for the production of a three-dimensional structure having a conical shape when D is 0 mm; under these conditions, the exposure time advantageously affects the shape and height of the structure thus obtained.

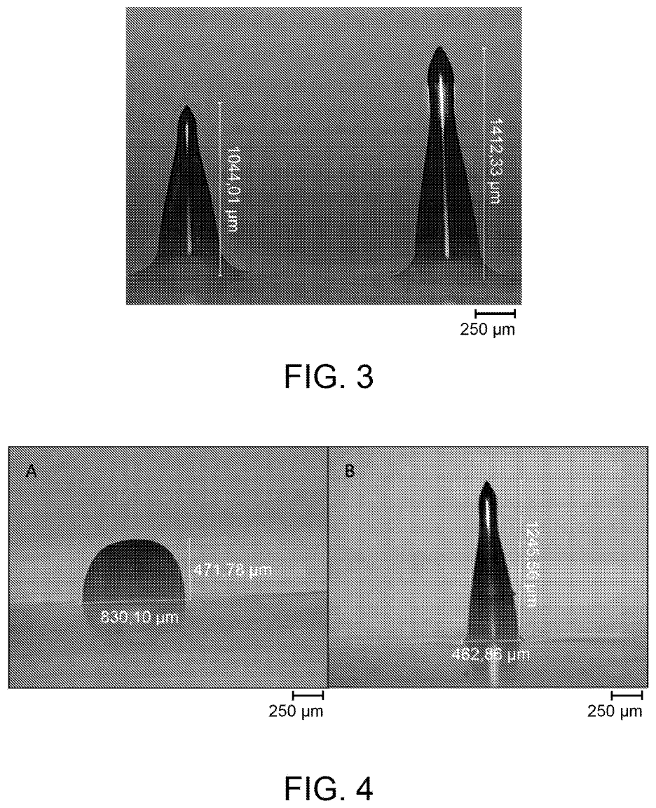

FIG. 3 shows two three-dimensional structures, particularly two needles, obtained with the process according to the present invention when the distance D is kept at 0 mm, increasing instead the exposure time from 15 seconds (three-dimensional structure on the left) to 45 seconds (three-dimensional structure on the right). It can be seen that the shape of the structure changes slightly with an increase in height of approximately 400 μm.

FIG. 4, instead, shows two three-dimensional structures (hemisphere and needle) obtained with the process according to the present invention, when: in FIG. 4a the distance D is kept at 2 mm, in FIG. 4b, instead, the distance D is kept at 0 mm and the irradiation time is 30 seconds. In the first case (FIG. 4a), the size of the base of the three-dimensional structure is about twice that of the three-dimensional structure in FIG. 4b (contact fibre, where there is less optical dispersion).

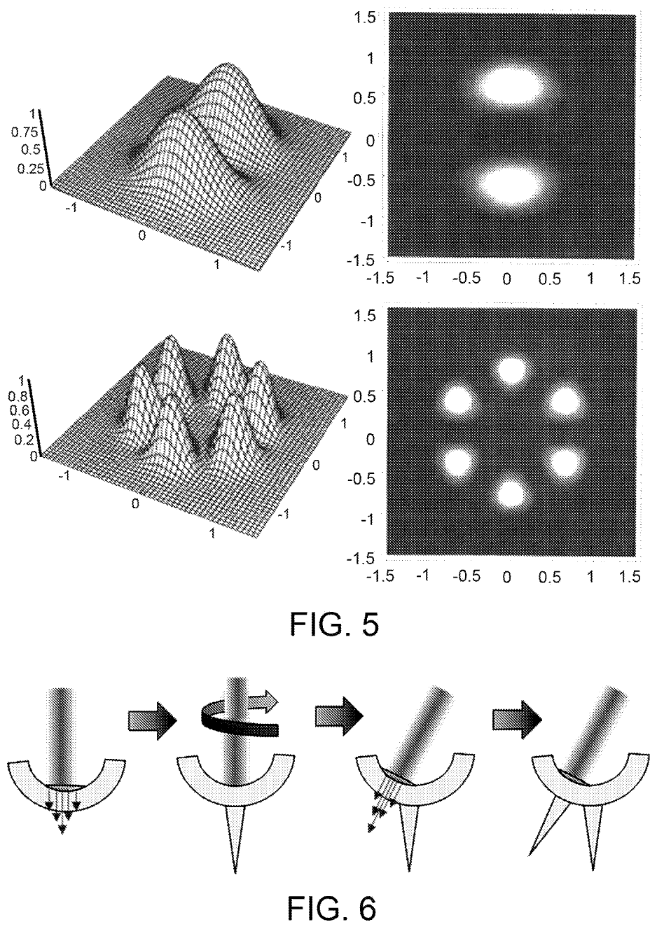

The three-dimensional structures obtainable with the process according to the present invention may have very different geometries, depending on the shape of the incident optical field which may, in turn, have a three-dimensional profile designed as desired, as shown in FIG. 5.

Advantageously, in fact, in the process according to the present invention, for each of the intensity profiles of the light coming out of the optical fibre, it is possible to obtain three-dimensional structures, which are asymmetrical, hollow, pyramidal, and so on.

Based on the same principle, it is also advantageously possible to create ordered sets of three-dimensional structures using automatically controlled bundles of optical fibres.

The process according to the present invention also advantageously makes it possible to produce three-dimensional structures on transparent surfaces that are not necessarily flat. In fact, the curvature of a surface is not a limitation for the process according to the present invention, as the optical fibre can be freely moved along the curvature of the surface. Advantageously, depending on the characteristics of the optical fibre used (i.e., the diameter and size of the core in which the light is propagated), it is possible to produce three-dimensional structures on surfaces having a smaller or larger radius of curvature. The latter, as well-known to the person skilled in the art, is defined by the equation: R=1/ρ where at each point P of a surface σ, the curve Γ of intersection between σ and any plane π containing the normal n to the surface in P has a given curvature ρ. As schematically depicted in FIG. 6, the optical fibre of the process according to the present invention can advantageously be moved along the curvature ρ of the surface and produce three-dimensional structures with arbitrary spacing.

According to an alternative embodiment of the process according to the present invention, the process also advantageously makes it possible to obtain a three-dimensional structure directly on the optical fibre when the latter is completely immersed in the photo-crosslinkable polymeric material. The resulting three-dimensional structure is preferably conical but may take on different geometries depending on the density and refractive index of the photo-crosslinkable polymeric material.

According to a preferred embodiment of the process according to the present invention, the photo-crosslinkable biocompatible hydrogels under consideration include hyaluronic acid methacrylate. As well-known to the person skilled in the art, hyaluronic acid (HA) is a linear polysaccharide consisting of repeating disaccharide units of glucuronic acid and N-acetyl glucosamine linked by 6β (1,4) and β (1,3) glycosidic linkages. Under physiological conditions it forms a negatively charged and highly hydrophilic sodium salt (sodium hyaluronate). HA chains can consist of 2,000-25,000 disaccharides corresponding to a relative molecular mass between 106-107 Da and a length of 2-25 μm. The resulting hyaluronic derivatives have physico-chemical properties that are significantly different from the native polymer, however most derivatives are biocompatible and biodegradable.

Hyaluronic acid methacrylate (MeHA) is a hyaluronic acid derivative with properties that can be modulated. The presence of methacrylic groups allows photo-crosslinking of HA derivatives. MeHA synthesis is preferably achieved by reaction with glycidyl methacrylate or methacrylic anhydride, which are then cross-linked via free radical polymerization when subjected to UV light (365 nm) in the presence of a photoinitiator.

Advantageously, the three-dimensional structures produced by the process according to the present invention can be made mechanically stronger through two approaches: either by inserting in the photo-crosslinkable polymeric material a di- or tetra-acrylate cross-linker, which allows the three-dimensional structure to maintain its shape even in an aqueous system; or by taking advantage of the principle of the double network by inserting in the photo-crosslinkable polymeric material a polymer capable of interacting through physical interactions with the other substances of the photo-crosslinkable polymeric material and a second polymer capable of forming a network by means of a radical reaction.

It is also possible to hypothesize the addition of non-photo-crosslinkable elements, as a passive (silica nanoparticles) or active (titania, zirconia, gold, silver and zinc nanoparticles) nanometric inorganic phase.

According to a preferred embodiment of the process according to the present invention, the at least one photoinitiator is preferably selected from: 2-hydroxy-2-methylpropiophenone, 2-hydroxy-4′-(2-hydroxyethoxy)-2-methylpropiophenone, lithium phenyl-2,4,6-trimethylbenzoylphosphinate (LAP).

LAP advantageously allows the potential UV damage to active molecules which could be present in the material to be reduced. LAP also offers greater water solubility, higher polymerization efficiency with a 365 nm light source, and minimal cytotoxicity. Additionally, LAP has significant absorbance above 400 nm, which allows for efficient polymerization using visible light. In other implementations, photoinitiators with absorbances at wavelengths in the visible spectrum could be employed.

In the process according to the present invention, the at least one non-photo-crosslinkable element can be selected from: functionalised nanoparticles, covalently HA-linked or free active ingredients or proteins, free proteins or active ingredients included in poly(lactic-co-glycolic) acid (PLGA) or other polymer microspheres. Ideally, these systems would allow for greater control of protein activity and stability compared to covalently directly linking the proteins to the polymer matrix.

Hyaluronic acid methacrylate used in the process according to the present invention may have a different degree of substitution (DS). Hyaluronic acid derivatization with polymerizable methacrylate residues, with precise control of the degree of substitution, can be achieved either in an aqueous environment with methacrylic anhydride excess relative to the HA hydroxyl groups, or with precise control of the DS in an aprotic solvent with glycidyl methacrylate (GMHA). As well-known to the person skilled in the art, the elastic modulus and dimensional stability of gels increase with the degree of substitution, which means that using MeHA with high DS can further reduce the percentage of diacrylate cross-linker in the photo-crosslinkable polymeric material.

2- or 4-Arm acrylate cross-linkers (polyethylene glycol diacrylate (PEGDA), 4-arm PEG-Acrylate, glycerol 1,3-diglycerolate diacrylate, tetra(ethylene glycol) diacrylate (TTE-GDA), di(ethylene glycol) diacrylate, bisphenol A glycerolate (1-glycerol/phenol) diacrylate, 1,6-hexanediol ethoxylate diacrylate, tricyclo[5. 2.1.02,6]decanedimethanol diacrylate), which give better mechanical properties to the three-dimensional structures obtained, can be incorporated in order to change the cross-linking density within the hydrogels and give the latter enhanced mechanical properties.

According to a preferred embodiment of the process according to the present invention, the photo-crosslinkable polymeric material may also include protected or unprotected active molecules, which will be incorporated into the three-dimensional structure during polymerization.

The three-dimensional structures obtained by the process according to the present invention may have a variety of uses, such as for example systems for the controlled release of drugs in the skin or eye.

Free or microsphere-embedded active molecules or proteins may be included in the photo-crosslinkable polymeric material during the cross-linking step. Said molecules may also be directly linked to the polymer matrix.

A further object of the present invention relates to a three-dimensional structure which can be obtained by using the process according to the present invention, as described above.

Another object of the present invention relates to the use of the three-dimensional structure which can be obtained by using the process according to the present invention, as described above.

In fact, the three-dimensional structure which can be obtained by using the process according to the present invention, as described above, can be advantageously used for:
administering active molecules via the dermal and/or transdermal route;
analysing target analytes in situ in tissues and cells;
sampling and analysing body fluids, such as blood, plasma, saliva, tears, interstitial body fluid, in both human and animal subjects.

### Example

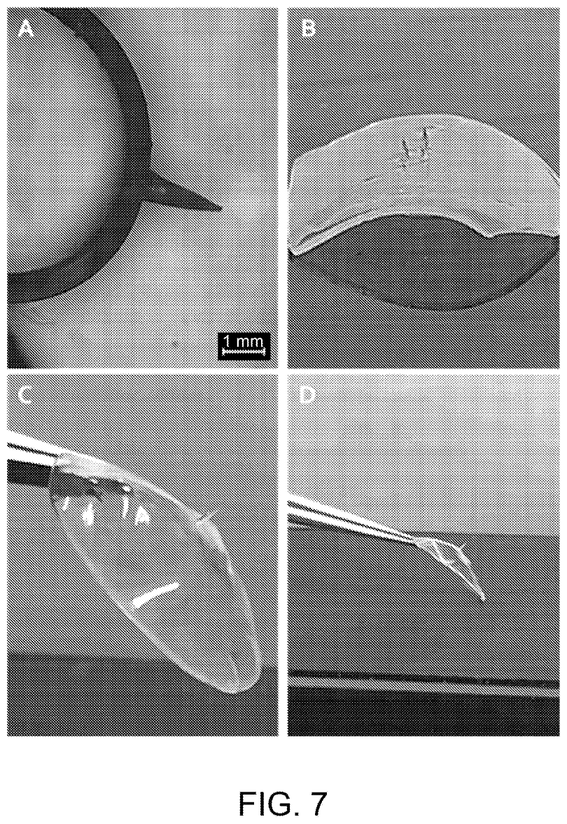

By way of example, FIG. 7 shows three-dimensional structures obtained on curved sur faces made of different materials by using the process according to the present invention. In FIG. 7a, a three-dimensional structure in the shape of an elongated cone made of a photo-crosslinked polymeric material by irradiation with light propagated by an optical fibre is obtained on a curved surface of polyethylene glycol acrylate. FIG. 7b shows two conical three-dimensional structures obtained on a curved surface of polypropylene (PP). In FIGS. 7a and b, the conical three-dimensional structures are obtained on rigid surfaces, whereas FIGS. 7c and d show examples of three-dimensional structures obtained on a highly flexible surface, in particular commercial contact lenses made of silicone hydrogel.

What is claimed is:

1. A process for producing a three-dimensional structure, the process comprising steps of:
a) providing a light source;
b) coupling said light source to a proximal end of at least one optical fibre, in such a way as to propagate a light generated by said light source through the at least one optical fibre and produce, at a distal end of the at least one optical fibre, a predetermined incident optical field, wherein said optical field maintains the same phase profile, the same spatio-temporal intensity, and the same frequency during propagation of the light through the at least one optical fibre and exit of the light from the at least one optical fibre;
c) providing at least one photo-crosslinkable polymeric material coated with at least one transparent material, wherein the at least one transparent material comprises a first surface placed in contact with the at least one photo-crosslinkable polymeric material and a second surface placed not in contact with the at least one photo-crosslinkable polymeric material and opposite to the first surface;
d) placing the distal end of the at least one optical fibre at a distance D from the second surface of the at least one transparent material, the distance D ranging from 0 mm to 5 mm; and
e) irradiating the second surface of the at least one transparent material with the light propagated and exiting the distal end of the at least one optical fibre for a period of time comprised between 1 second and 5 minutes, so as to obtain propagation of the light through the at least one transparent material towards the at least one photo-crosslinkable polymeric material and to obtain photo-crosslinking, by irradiation, of the polymeric material thus irradiated, with consequent formation of the three-dimensional structure.

2. The process of claim 1, wherein the first surface of the at least one transparent material is a curved surface, and wherein the at least one transparent material is selected from: transparent natural polymer, transparent synthetic polymer, transparent pre-polymerized photo-crosslinked polymer, transparent polyethylene terephthalate, transparent polypropylene, transparent glass, transparent hydrogel, and transparent silicone hydrogel.

3. The process of claim 1, wherein the at least one photo-crosslinkable polymeric material comprises: at least one photo-crosslinkable biocompatible hydrogel or mixtures thereof, at least one photoinitiator and optionally at least one non-photo-crosslinkable element.

4. The process of claim 3, wherein the at least one photo-crosslinkable biocompatible hydrogel comprises at least one of: hyaluronic acid acrylate derivatives, hyaluronic acid acrylate, hyaluronic acid methacrylate, acrylate or methacrylate gelatin derivatives, gelatin-methacryloyl (GelMA), or mixtures thereof.

5. The process of claim 4, wherein the at least one photo-crosslinkable biocompatible hydrogel further comprises at least one of: di- or tetra-acrylate cross-linker, 2- or 4-arm acrylate (polyethylene glycol diacrylate (PEGDA), 4-arm PEG-Acrylate, glycerol 1,3-diglycerolate diacrylate, tetra(ethylene glycol) diacrylate (TTEGDA), di(ethylene glycol) diacrylate, bisphenol A glycerolate (1-glycerol/phenol) diacrylate, 1,6-hexanediol ethoxylate diacrylate, tricyclo[5. 2.1.02,6]decanedimethanol diacrylate).

6. The process of claim 3, wherein the at least one photoinitiator is selected from: 2-hydroxy-2-methylpropiophenone, 2-hydroxy-4′-(2-hydroxyethoxy)-2-methylpropiophenone, and lithium phenyl-2,4,6-trimethylbenzoylphosphinate (LAP).

7. The process of claim 3, wherein the at least one non-photo-crosslinkable element is selected from: silk fibroin, silica nanoparticle, titania nanoparticle, zirconia nanoparticle, gold nanoparticle, silver nanoparticle, zinc nanoparticle, substance having the biological activity to act as a pharmaceutical active ingredient, functionalized nanoparticle, free protein, protein covalently linked to hyaluronic acid methacrylate, and substance having the biological activity to act as a pharmaceutical active ingredient included in a poly(lactic-co-glycolic acid) microsphere.

8. The process of claim 1, wherein in step e) the period of time is comprised between 10 seconds and 60 seconds.

9. The process of claim 1, wherein in step d) the distance D is comprised between 0 mm and 1 mm.

10. The process of claim 1, wherein in step b) the light source is coupled to a proximal end of a bundle of optical fibres, wherein said bundle comprises a number of optical fibres comprised between 2 and 1000, and wherein in said bundle the optical fibres are arranged at a mutual distance comprised between 0 mm and 10 mm, so as to control interference of relative optical fields of each optical fibre.
